(19) United States
(12) Patent Application Publication
(10) Pub. No.: US 2021/0369880 A1
(43) Pub. Date: Dec. 2, 2021

(54) Assembly for dispensing sanitiser fluid

(71) Applicant: E Industrial Engineering Pty Ltd, Wetherill Park (AU)

(72) Inventor: David NEWELL, Mona Vale NSW (AU)

(21) Appl. No.: 17/003,561

(22) Filed: Aug. 26, 2020

(30) Foreign Application Priority Data
May 27, 2020 (AU) 2020100864

(51) Int. Cl. A61L 2/00; B05B 11/00

(52) U.S. Cl. CPC A61L 2/0088; A61L 2202/15; B05B 11/3052

(57) ABSTRACT

An assembly 10 for dispensing sanitiser fluid. The assembly 10 includes a post 12 having opposed ends 14, 16, a pedal 18 pivotally securable adjacent one of the ends 16 of the post 12, a platform 20 dimensioned to be received within the post 12, the platform 20 configured to support a reservoir containing the sanitiser fluid, an elongate member 22 dimensioned to extend between the pedal 18 and the platform 20 such that pivoting the pedal 18 moves the elongate member 22 to cause the platform 20 to be displaced along the post 12, a stop 24 securable to the post 12 to be operatively above the platform 20 such that, in use, the reservoir is urged against the stop 24 when the platform 20 is displaced to cause sanitiser fluid to be dispensed from the reservoir, and an open-topped receptacle 26 defining an internal space 28, the receptacle 26 securable to the post 12 to be operatively below the stop 24 to allow collecting dispensed fluid in the internal space 28.

### TECHNICAL FIELD

The present disclosure relates generally to assemblies for dispensing fluid and particularly relates to assemblies for dispensing antimicrobial hand sanitiser fluid.

### BACKGROUND

Antimicrobial hand sanitisers are widely used in many different environments to reduce the risk of spreading pathogens. For example, it is commonplace in hospitals for a hand sanitiser dispenser to be mounted at an end of each patient's bed, and at each ward entrance/exit, to encourage frequent application of hand sanitiser by medical practitioners.

Typical hand sanitiser dispensers comprise a vessel containing sanitiser fluid and a pump-action closure mechanism. Use involves a user manually depressing the pump to dispense a portion of the fluid. This arrangement presents an infection risk as users must touch the closure mechanism during each use, meaning that the dispenser is a high risk site for collecting pathogens.

Past approaches to address this issue have involved providing electrically-powered sanitiser dispensers mounted to a surface, such as a wall, or a post. These devices include a mechanism to detect the presence of a user's hand within range and automatically dispense a portion of sanitiser fluid. However such devices often result in sanitiser fluid bypassing a user's hands and spilling on to walls, the floor and/or the user's clothing. Furthermore the requirement for electrical power means that such devices require regular maintenance to replace batteries.

Any discussion of documents, acts, materials, devices, articles or the like which has been included in the present specification is not to be taken as an admission that any or all of these matters were common general knowledge in the field relevant to the present disclosure as it existed before the priority date of each of the appended claims.

### SUMMARY

According to at least one disclosed embodiment, there is provided an assembly for dispensing sanitiser fluid. The assembly includes a post having opposed ends, a pedal pivotally securable adjacent one of the ends of the post, a platform dimensioned to be received within within the post, the platform configured to support a reservoir containing the sanitiser fluid, an elongate member dimensioned to extend between the pedal and the platform such that, in use, pivoting the pedal moves the elongate member to cause the platform to be displaced along the post, a stop securable to the post to be operatively above the platform such that, in use, the reservoir is urged against the stop when the platform is displaced to cause sanitiser fluid to be dispensed from the reservoir, and an open-topped receptacle defining an internal space, the receptacle securable to the post operatively below the platform to allow collecting dispensed fluid in the internal space.

The post may include a mounting structure extending from a sidewall, and the receptacle is configured to be releasably mounted on the mounting structure.

The mounting structure may include at least one pin, and the receptacle may include a complementary at least one aperture.

The receptacle may include a base defining a peripheral region and a shroud arranged at least partially around the base to extend away from the base, and the shroud is at least partially transparent.

The elongate member may include an inner member arranged within a sleeve, the inner member having an abutment feature arranged to abut an end of the sleeve, the abutment feature being movable along the inner member.

The inner member may be threaded, and the abutment feature is a nut threadedly engaged with the inner member.

The stop may include a bar configured to be releasably secured through the post.

The stop may include a cap configured to be mounted to one of the ends of the post.

The assembly may also include a base mountable to one of the ends of the post, and the pedal is pivotally securable to the base.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

It will be appreciated embodiments may comprise steps, features and/or integers disclosed herein or indicated in the specification of this application individually or collectively, and any and all combinations of two or more of said steps or features.

### BRIEF DESCRIPTION OF DRAWINGS

Embodiments will now be described by way of example only with reference to the accompany drawings in which:

FIG. 1 is a perspective view of a first embodiment of an assembly for dispensing sanitiser fluid;

FIG. 2 is a perspective detail view of the assembly shown in FIG. 1;

FIG. 3 is side view of the assembly shown in FIGS. 1 and 2;

FIG. 4 is a side view of the assembly shown in FIGS. 1 to 3 with a fluid receptacle removed;

FIGS. 5 and 6 are perspective and side views, respectively, of a dispensing mechanism which forms part of the assembly shown in the previous figures; and

FIGS. 7 and 8 are side and front views, respectively, of a second embodiment of an assembly for dispensing sanitiser

### DESCRIPTION OF EMBODIMENTS

In the drawings, reference numeral 10 generally designates an assembly 10 for dispensing sanitiser fluid. The assembly 10 includes a post 12 having opposed ends 14, 16, a pedal 18 pivotally securable adjacent one of the ends 16 of the post 12, a platform 20 dimensioned to be received within the post 12, the platform 20 configured to support a reservoir (not illustrated) containing the sanitiser fluid, an elongate member 22 dimensioned to extend between the pedal 18 and the platform 20 such that pivoting the pedal 18 moves the elongate member 22 to cause the platform 20 to be displaced along the post 12, a stop 24 securable to the post 12 to be operatively above the platform 20 such that, in use, the reservoir is urged against the stop 24 when the platform 20 is displaced to cause sanitiser fluid to be dispensed from the reservoir, and an open-topped receptacle 26 defining an internal space 28, the receptacle 26 securable to the post 12 to be operatively below the stop 24 to allow collecting dispensed fluid in the internal space 28.

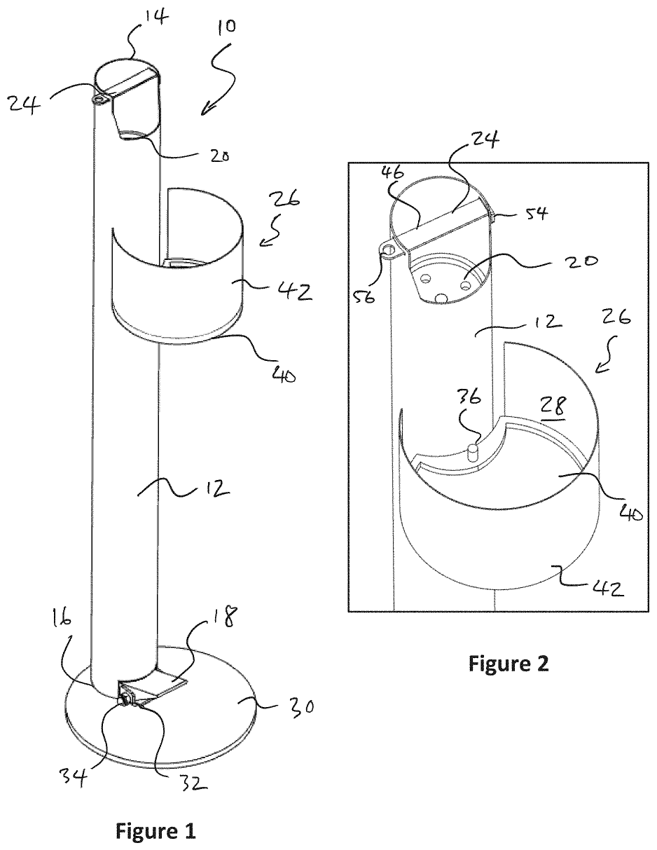

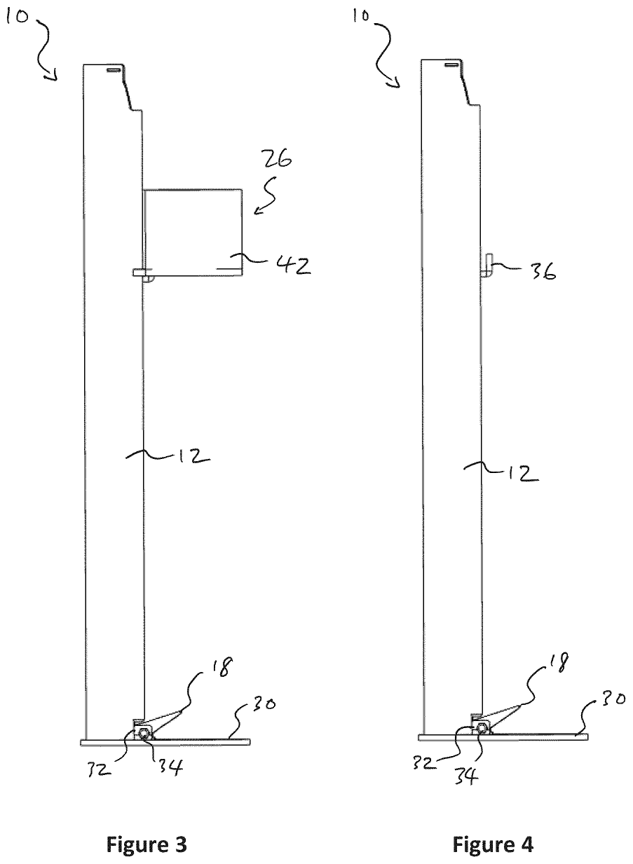

FIGS. 1 to 6 illustrate a first embodiment of the assembly 10. In this embodiment the assembly also includes a base, in the form of a plate 30, configured to be secured to one of the ends 16 of the post 12 to allow supporting the post 12 on a surface, such as the floor. In some embodiments (not illustrated) the plate 30 defines a plurality of apertures to allow securing the plate 30 to the surface, such as with fasteners. In other embodiments (not illustrated) the base comprises a moulded or cast structure shaped to enhance stability of the post 12.

In the illustrated embodiment the pedal 18 is pivotally securable to the plate 30 via a bracket 32 configured to retain a fastener 34 which defines the pivot. In other embodiments (not illustrated) the base includes an integrated pivot.

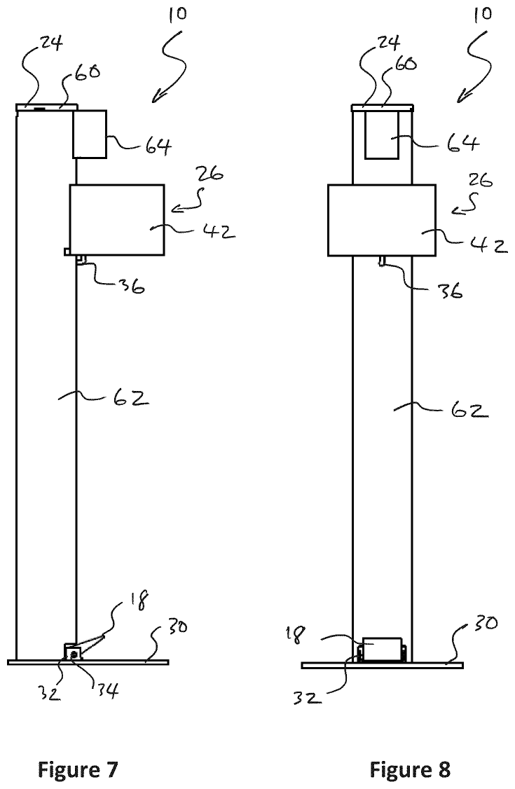

The post 12 is a hollow cylinder, typically formed from stainless steel. The post 12 is dimensioned according to the reservoir it is configured to be used in conjunction with. For example, in the embodiment illustrated in FIGS. 1 to 5 the post 12 is configured to receive a specific bottle-shaped reservoir, and defines an internal diameter of 4 inches to allow receiving the bottle. In the embodiment illustrated in FIGS. 6 and 7 the post 62 defines an internal diameter of 5 inches to allow receiving an alternative bottle.

Best shown in FIG. 4, the post 12 has a mounting structure, in the illustrated embodiment in the form of a bent pin 36, secured to a sidewall 38. The pin 36 has a free end arranged to extend parallel to the sidewall 38 to allow supporting the receptacle 26 relative to the post 12. In other embodiments (not illustrated) the mounting structure comprises one or more alternatively configured pins or plates to which the receptacle 26 is securable. In yet other embodiments (not illustrated) the mounting structure comprises one or more apertures or recesses defined in the sidewall 38 and configured to allow the receptacle 26 to engage the post 12.

Best shown in FIG. 2, the receptacle 26 is releasably mountable on the pin 36. The receptacle 26 includes a drip tray 40 which defines an aperture dimensioned to receive the pin 36. In some embodiments (not illustrated) the drip tray 40 also defines one or more drainage apertures to allow draining sanitiser fluid collected in the receptacle 26.

The drip tray 40 defines a peripheral region and has a shroud 42 arranged partially around the peripheral region to extend away from the drip tray 40. The internal space is defined between the shroud 42 and the sidewall 38 of the post 12. It will be appreciated that the shroud 42 may be arranged around more or less of the peripheral region. Typically the shroud 42 is at least partially, or entirely, transparent to allow viewing inside the receptacle 26.

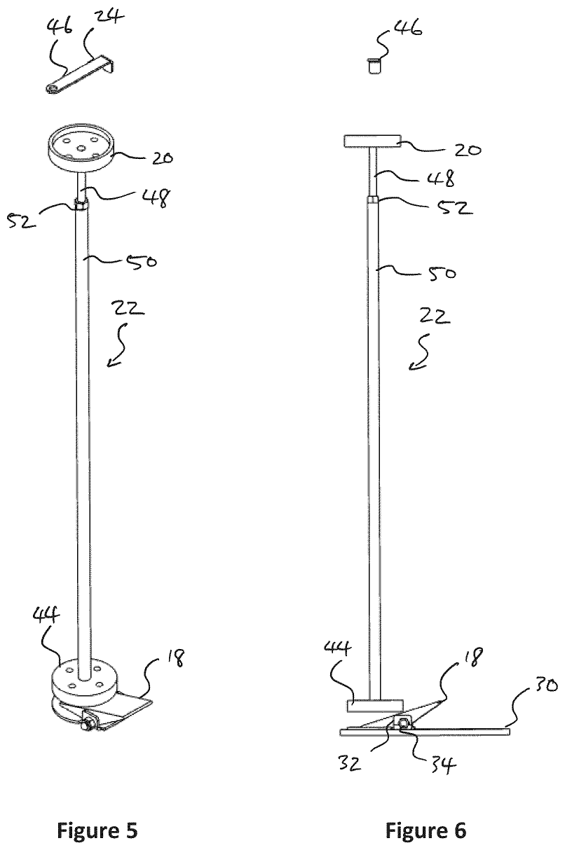

FIGS. 5 and 6 illustrate the dispensing mechanism in isolation. The mechanism includes the pedal 18, the elongate member 22 arranged so that one end rests on the pedal 18, the platform 20 secured to the other end of the elongate member 22, and the stop 24, in this embodiment in the form of a bar 46, arranged to be operatively above the platform 20. The platform 20 is shaped and dimensioned to closely fit within the post 12. In this embodiment the platform 20 is dimensioned to slidably engage an inner wall of the post 12.

The elongate member 22 comprises a sub-assembly configured to allow adjusting a length of the member 22. The sub-assembly includes an inner member 48 arranged within a sleeve 50 and a block 44 secured to an end of the sleeve 50. An abutment feature, in the illustrated embodiment in the form of a nut 52, is releasably secured to the inner member 48 to abut the other end of the sleeve 50. Adjusting the position of the abutment feature along the inner member 48 adjusts the effective length of the elongate member 22, This allows accommodating taller or shorter reservoirs between the platform 20 and the stop 24. In this embodiment the inner member 48 defines a thread and the nut 52 is threadedly engaged with the inner member 48.

Best shown in FIG. 2, the bar 46 is configured to be arranged to extend through slots defined in the sidewall 38 of the post 12. One end of the bar 46 has a flange 54 arranged to interfere with the sidewall 38. The other end of the bar 46 defines a hole 56 for receiving a padlock or cable to inhibit the bar 46 being removed from the post 12. This also allows inhibiting the reservoir being removed from the post 12.

FIGS. 7 and 8 show a second embodiment of the assembly 10 which shares many features with the first embodiment, whereby common reference numerals indicate common features. In this embodiment the stop 24 is in the form of a cap 60 configured to be secured to, and cover, an end of the post 62. A second shroud 64 is securable to the post 12 to be adjacent to the cap 60 and cover a dispensing nozzle (not illustrated) of the reservoir.

Using the assembly 10 to dispense sanitiser fluid requires a user to depress the pedal 10 with its toot. Depressing the pedal 18 causes the pedal 18 to pivot about the fastener 34 to urge against the block 44. This transmits force to the elongate member 22 which, in turn, displaces the platform 20 operatively upwards. Moving the platform 20 elevates the reservoir to push against the stop 24 to cause sanitiser fluid to be dispensed from the reservoir. Typically, the reservoir includes a pump-action closure and therefore moving the platform 20 presses the closure against the stop 24 to cause fluid to be emitted. Fluid which is not caught by the user's hands is received in the receptacle 26. The receptacle 26 is demountable from the post 12 to allow removal of waste fluid and cleaning.

The assembly 10 is configured to dispense sanitiser fluid from a reservoir by a user operating a pedal 18, This allows ‘hands-free’ dispensing of the fluid. Fluid which is not caught by the user's hands is received in the receptacle 26. This advantageously inhibits fluid being sprayed onto the user's clothing, the floor, or, where the user is a child, being sprayed on or near its face.

It will be appreciated by persons skilled in the art that numerous variations and/or modifications may be made to the above-described embodiments, without departing from the broad general scope of the present disclosure. The present embodiments are, therefore, to be considered in all respects as illustrative and not restrictive.

1. An assembly for dispensing sanitiser fluid, the assembly including:
a post having opposed ends;
a pedal pivotally securable adjacent one of the ends of the post;
a platform dimensioned to be received within the post, the platform configured to support a reservoir containing the sanitiser fluid;
an elongate member dimensioned to extend between the pedal and the platform such that, in use, pivoting the pedal moves the elongate member to cause the platform to be displaced along the post;
a stop securable to the post to be operatively above the platform such that, in use, the reservoir is urged against the stop when the platform is displaced to cause sanitiser fluid to be dispensed from the reservoir; and
an open-topped receptacle defining an internal space, the receptacle securable to the post to be operatively below the stop to allow collecting dispensed fluid in the internal space.

2. The assembly of claim 1, wherein the post includes a mounting structure extending from a sidewall, and the receptacle is configured to be releasably mounted on the mounting structure.

3. The assembly of claim 2, wherein the mounting structure includes at least one pin, and the receptacle includes a complementary at least one aperture.

4. The assembly of claim 1, wherein the receptacle includes a base defining a peripheral region and a shroud arranged at least partially around the base to extend away from the base, and wherein the shroud is at least partially transparent.

5. The assembly of claim 1, wherein the elongate member includes an inner member arranged within a sleeve, the inner member having an abutment feature arranged to abut an end of the sleeve, the abutment feature being movable along the inner member.

6. The assembly of claim 5, wherein the inner member is threaded, and the abutment feature is a nut threadedly engaged with the inner member.

7. The assembly of claim 1, wherein the stop includes a bar configured to be releasably secured through the post.

8. The assembly of claim 1, wherein the stop includes a cap configured to be mounted to one of the ends of the post.

9. The assembly of claim 1, further including a base mountable to one of the ends of the post, and wherein the pedal is pivotally securable to the base.
